(19) United States
(12) Patent Application Publication
(10) Pub. No.: US 2007/0100244 A1
(43) Pub. Date: May 3, 2007

(54) Wrist-worn monitor for heartbeat detection

(76) Inventor: Chin-shu Lin, Tainan Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/261,459

(22) Filed: Oct. 31, 2005

(51) Int. Cl. A61B 5/02

(52) U.S. Cl. 600/503

(57) ABSTRACT

A wrist-worn monitor for heartbeat detection includes a detecting device provided on a belt. The detecting device comprises a first detecting element on one side and a pair of second detecting elements on another side. When the belt is worn on a wrist of a user, the first detecting element will be in touch with a wrist of a user while the user presses the second detecting elements with two fingers of another hand to get the reading of his/her heartbeat.

### BACKGROUND OF THE INVENTION

1. Field of the Invention

This invention relates to a wrist-worn monitor for heartbeat detection, in particular to a monitor worn on a wrist to get the reading of heartbeat easily.

2. Background of the Related Prior Art

A conventional heartbeat monitor currently used on an exercising or medical equipment has become to be wireless, which is convenient in use. As shown in FIG. 5, a monitor A comprises two detecting sections A1 extending from respective sides. Each detecting section A1 has a detecting terminal A11 and a locking hole A12 at the endmost. A strap B comprises a buckle B1 at the endmost to connect with the locking hole A12 to secure the strap B to the chest of a person with the detecting sections A1 of the monitor A being in touch with the front of the person's chest so as to monitor the heartbeat of that person. The signal from the monitor A is transmitted to a receiver C adjacent to the monitor A.

However, the monitor has some shortcomings:
1. After exercise, the person's breath will increase, which makes the user difficult in breath.
2. When a female uses the monitor, the monitor will have difficulty to be in contact with the chest for monitoring.
3. The buckle will cause the user uncomfortable, in particular when they are doing exercise.

### SUMMARY OF THE INVENTION

It is the primary object of the present invention to provide a wrist-worn monitor for heartbeat detection, which is worn to a users' wrist so that the user has no uncomfortable feeling.

It is another object of the present invention to provide a wrist-worn monitor for heartbeat detection, which is light in weight and easy to operate.

The present invention includes a detecting device provided on a belt. The detecting device comprises a first detecting element on one side and a pair of second detecting elements on another side. When the belt is worn on a wrist of a user, the first detecting element will be in touch with the skin while the user presses the second detecting elements with two fingers of another hand to get the reading of his/her heartbeat.

### BRIEF DESCRIPTION OF THE DRAWINGS

FIG. 1 is a perspective view of the present invention;

FIG. 2 is a circuit diagram of the present invention;

FIG. 3 is a perspective view of the present invention in an operating status;

FIG. 4 is a perspective view of a second embodiment of the present invention, and

FIG. 5 is perspective view of a conventional heartbeat monitor in an operating status.

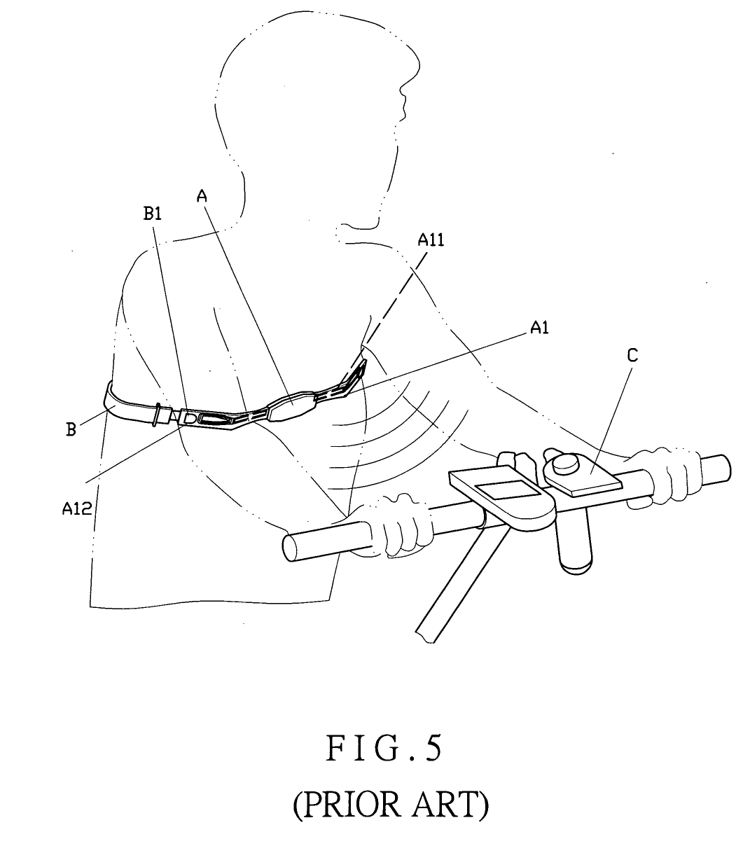

### DETAILED DESCRIPTION OF THE PREFERRED EMBODIMENT

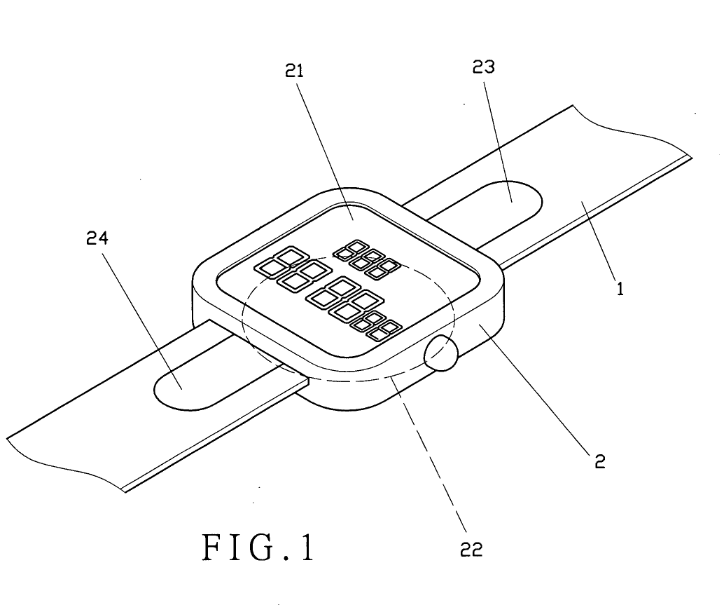

As shown in FIG. 1, the present invention comprises a belt 1 and a detecting device 2.

The belt 1 is worn to a user's wrist. The detecting device 2 is provided on the belt 1. The detecting device 2 comprises a screen 21 on one side and a first detecting element 22 on the other side thereof. A pair of second detecting elements 23 and 24 connected with the detecting device 2 is provided on the belt 1.

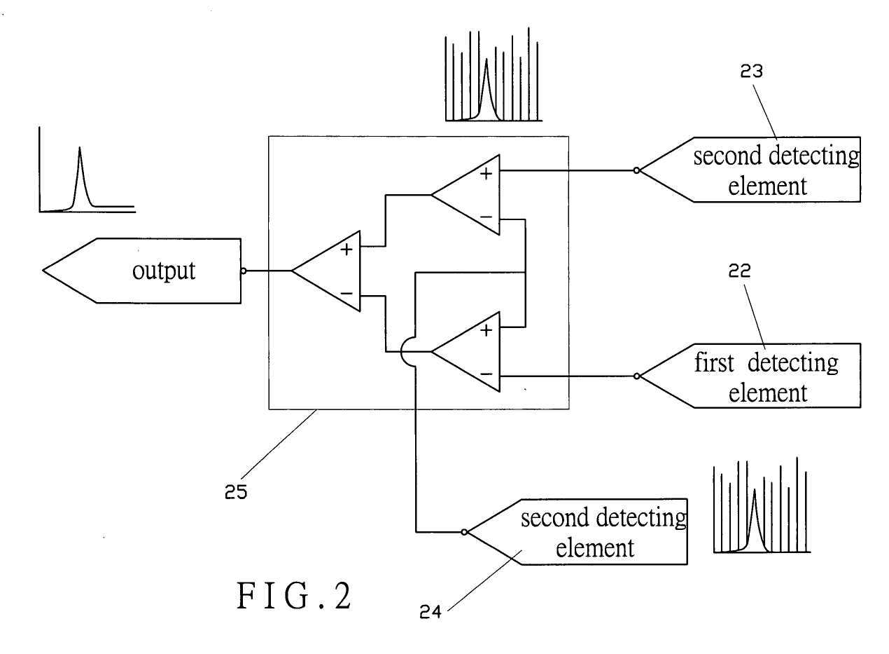

As shown in FIG. 2, the detecting device 2 comprises a difference amplifier 25 therein. The difference amplifier 25 has contacts connected to the first detecting element 22 and the second detecting elements 23 and 24, respectively, so that all signals detected by the contacts will be transformed to a digit to show on the screen 21 of the detecting device 2.

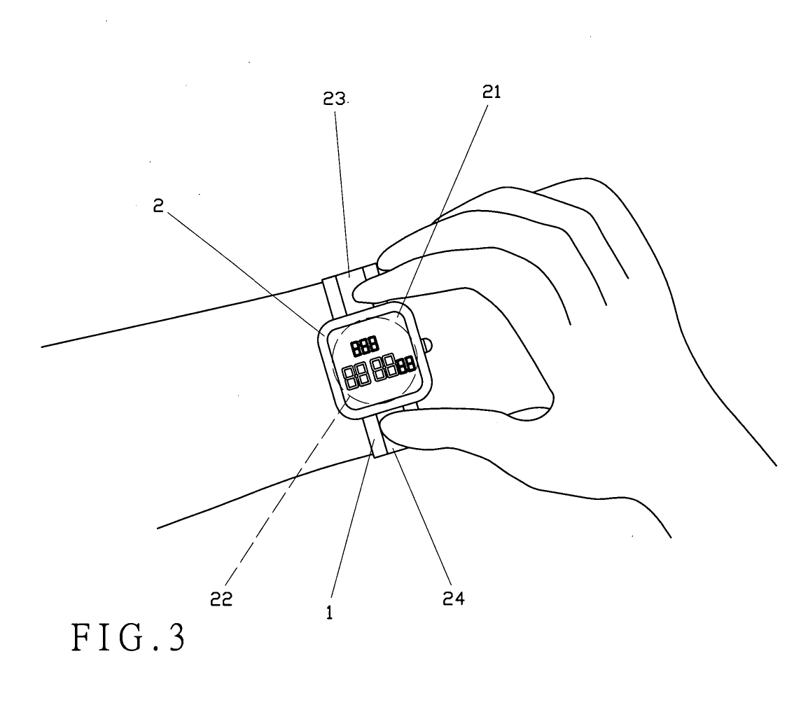

To operate the present invention, as shown in FIG. 3, the belt 1 is worn on a user's wrist with the first detecting element 22 in touch with the skin. The user uses two fingers of the other hand to press the second detecting elements 23 and 24. This constitutes a circuit. A mini current will flow through the two hands to the user's chest to measure the heartbeat and then send the result to the difference amplifier 25 of the detecting device 2. The difference amplifier 25 filters out inadequate signal and sends the frequency in a pulse wave to the detecting device 2 to be transformed to a digit for showing on the screen 21.

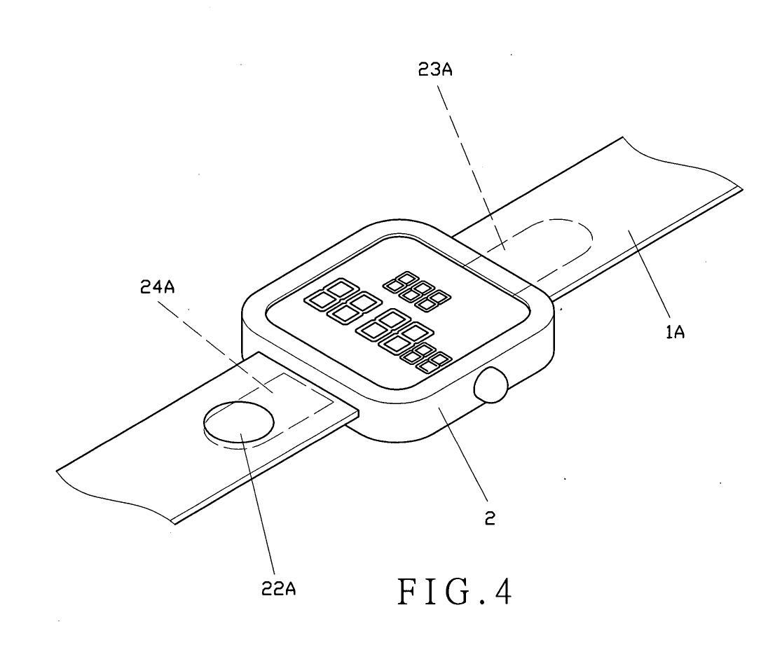

The first detecting element 22, as described above, is designed to be in touch with the wrist. FIG. 4 shows a second embodiment of the present invention. Two detecting elements 23A and 24A are provided at the bottom of a bolt 1A to be in touch with the wrist, while the user uses one finger to press a first detecting element 22A provided on the belt 1A. The result will be the same as the first embodiment as descried above.

What is claimed is:

1. A wrist-worn monitor for heartbeat detection comprising:
a belt;
a detecting device disposed on said belt, said detecting device comprising a first detecting element on one side and two second detecting elements on another side;
said first detecting element being in touch with one wrist of a user, while the user pressing said two second detecting elements with another hand to detect the heartbeat.

2. The wrist-worn monitor for heartbeat detection, as recited in claim 1, wherein said two second detecting elements are provided on said belt, extending from said detecting device along said belt, respectively.

3. The wrist-worn monitor for heartbeat detection, as recited in claim 1, wherein said detecting device comprises a difference amplifier to output signals received from said first detecting element and said second detecting elements.
